(19) United States
(12) Patent Application Publication
(10) Pub. No.: US 2023/0293319 A1
(43) Pub. Date: Sep. 21, 2023

(54) FOOT COVER FOR PROSTHETIC FOOT

(71) Applicant: Össur Iceland ehf, Reykjavík (IS)

(72) Inventors: Bjarni Andresson, Reykjavík (IS); Larus Gunnsteinsson, Reykjavík (IS); Jeroen Nijman, Reykjavík (IS); Felix Starker, Reykjavík (IS); Aron Kristbjorn Albertsson, Reykjavík (IS); Maria Gudrun Sveinbjornsdottir, Reykjavík (IS)

(21) Appl. No.: 18/180,757

(22) Filed: Mar. 8, 2023

Related U.S. Application Data
(60) Provisional application No. 63/269,533, filed on Mar. 17, 2022.

(51) Int. Cl. A61F 2/66

(52) U.S. Cl. CPC A61F 2/66; A61F 2002/5001

(57) ABSTRACT

A foot cover for a prosthetic foot can allow a user to more easily remove the foot cover when the foot cover needs to be switched out or cleaned. The foot cover can have a hinge near a heel end of the foot cover. The hinge can allow a user to rotate a section of the foot cover to increase the size of the opening so that the foot prosthetic can be inserted into or removed from an inner cavity of the foot cover. The foot cover can be an easily separable two-part foot cover, the two parts coupled together with magnets or another mechanical fastener.

### INCORPORATION BY REFERENCE TO ANY PRIORITY APPLICATIONS

This application claims priority to U.S. Provisional Application No. 63/269,533, filed Mar. 17, 2022, which is incorporated herein by reference. Any and all applications for which a foreign or domestic priority claim is identified in the Application Data Sheet as filed with the present application are hereby incorporated by reference under 37 CFR 1.57.

### BACKGROUND

### Field

The present application relates to foot covers in general, and more particularly, to two-part foot covers configured to allow a user to easily put on and remove the foot cover.

### Description of the Related Art

Conventional prosthetic foot designs are sometimes enclosed in a cosmesis or foot cover to improve the aesthetic appeal of the prothesis. During use, water, dirt, or other debris may become trapped inside the foot cover, requiring removal of the foot cover in order to clean the foot cover. Conventional designs have the shape of a human foot and are made, for example, of rubber or other flexible materials. Although these materials are flexible, the foot cover must stay on during use, so foot covers are intentionally difficult to remove.

Accordingly, there is a need for an improved foot cover that can stay on during use but can be easily removed for cleaning or simply switching foot covers.

### SUMMARY

Foot covers, also known as cosmeses, for prosthetic feet are typically manufactured out of one piece of material. On the one hand, although the material may be flexible, the foot cover must stay on during use in order for the foot cover to be useful. Therefore, foot covers are typically difficult to remove, and strenuous effort may be required to remove a foot cover from a prosthetic foot. On the other hand, a user may need to change foot covers for a multitude of reasons. Different foot covers may have different properties based on which activity they are best used for. A foot cover may include specific features for different activities or shoe types, such as an alignment of a heel of the foot cover to a shoe heel height, and a user may need to switch between different foot covers when engaging in different activities, for example, wearing a prosthetic foot cover designed for daily use and switching to a prosthetic foot cover designed for running when participating in physical exercise. Additionally, a user may need to change the prosthetic foot they are using, and a user may need to put the foot cover on a different foot. Furthermore, after usage, the foot cover may get dirt or debris trapped inside, therefore, the foot cover may need to be removed in order to be cleaned.

It is desirable to have a foot cover that stays on the prosthetic foot during activity but may be easily removed by the user when the foot cover needs to be switched out or cleaned.

In some configurations, the foot cover may include a first section, and a second section configured to be removably coupled to the first section to form the foot cover that may include a sole portion and a top portion connected to the sole portion along a periphery of the sole portion to define an inner cavity configured to receive the prosthetic foot, an upper edge of the top portion forming an opening leading to the inner cavity, wherein the first and second sections can form a releasable connection at a location near the upper edge of the top portion.

In some configurations, the foot cover may be made of a waterproof material.

In some configurations, the waterproof material may include EVA, polyurethane, TPU, injected rubber, or compressed rubber.

In some configurations, the foot cover may include at least one indentation or ring on an outer surface of the foot cover.

In some configurations, the foot cover may include a first section including a first inner surface, and a portion of the first inner surface including at least one groove.

In some configurations, the foot cover may include at least one fastener configured to releasably couple the second section to the first section.

In some configurations, the at least one fastener can be on an outer surface of the foot cover.

In some configurations, the at least one fastener can be on an inner surface of the foot cover.

In some configurations, the at least one fastener can include a clasp, snap, magnet, button, hook-and-loop fastener, an indention and raised surface, or an elastic band and hook.

In some configurations, the prosthetic foot may include a grip pad at one or both of a heel portion or a toe portion of the foot prothesis.

In some configurations, the foot cover may include: a first section; a second section rotatably coupled to the first section at a hinge to form the foot cover that can include a sole portion and a top portion connected to the sole portion along a periphery of the sole portion to define an inner cavity configured to receive the prosthetic foot, an upper edge of the top portion forming an opening leading to the inner cavity; and at least one fastener configured to couple the first section to the second section at a second location away from the hinge, wherein the first section can include a front section and the second section can include a heel section.

In some configurations, the hinge may include a live hinge.

In some configurations, the sole portion may include at least one groove on an inner surface of the sole portion, wherein the at least one groove may be configured to guide water or debris out of the foot cover.

In some configurations, the at least one fastener may include a clasp, snap, magnet, button, hook-and-loop fastener, an indention and raised surface, or an elastic band and hook.

In some configurations, the second section may include a raised portion on an inner surface of the second portion, the raised portion may be configured to secure a heel portion of the prosthetic foot when the prosthetic foot is inserted into the first section.

In some configurations, the at least one fastener may include first and second components, the first component located on the first section and the second component located on the second section.

In some configurations, the first component may be coupled to a first outer surface of the first section, and the second component may be coupled to a second outer surface of the second section.

In some configurations, the first component may be coupled to a first inner surface of the first section, and the second component may be coupled to a second inner surface of the second section.

In some configurations, the second location may be more anterior than a location of the hinge.

In some configurations, the first and second sections may include overlapping parts when the second section is fastened to the first section.

In some configurations, the foot cover may include: a bottom section configured to receive the prosthetic foot, the bottom section may include a top opening; and a top section configured to cover a top of the prosthetic foot, and secure the prosthetic foot in the bottom section, the top section may include a bottom opening, wherein the top opening may be removably coupled to the bottom opening.

In some configurations, the bottom section may include an inner surface, and wherein the inner surface may include at least one groove configured to guide water or debris out of the cosmesis.

In some configurations, the top opening and the bottom opening may each include a perimeter, each perimeter may include one component of a fastener.

In some configurations, the fastener may include a quick release fastener.

In some configurations, the perimeter of the top opening may include a first one or more magnets, and the perimeter of the bottom opening may include a second one or more magnets, the first and second one or more magnets may have opposite polarities.

In some configurations, a bottom surface of the foot cover may include one or more grip features.

In some configurations, the grip features may extend over a portion of the bottom surface.

In some configurations, the grip features may extend over the entire bottom surface.

In some configurations, the grip features may include one or more of: a sandblasted piece of material coupled to the bottom surface; a plurality of cuts, micro cuts, or indentations on the bottom surface; and/or a tread pattern.

In some configurations, an arch region of the bottom surface may not include any grip features.

In some configurations, the grip features may cover a toe portion of the bottom surface.

In some configurations, the overlapping parts of the first and second sections may form a seal.

In some configurations, the seal may include a curved configuration.

In some configurations, the seal may curve upward from a bottom of the seal to a top of the seal.

In some configurations, the seal may curve rearwardly from a bottom of the seal to a top of the seal.

In some configurations, a top of the seal may form a first angle with a horizontal plane, and a bottom of the seal may form a second angle with the horizontal plane, and the first angle may be larger than the second angle.

In some configurations, the first section may include a tapered portion.

In some configurations, the tapered portion may include a portion of the first section near the seal, wherein a thickness of the tapered portion may increase from a thickness of the overlapping part of the first section to a thickness of the first section.

In some configurations, the tapered portion may include a triangular shape.

In some configurations, the tapered portion may include a width, and the width may increase from a bottom of the tapered portion to a top of the tapered portion.

In some configurations, the fastener may include a hole in the overlapping part of the second section and an elastic cord coupled to the overlapping part of the first section, wherein the elastic cord extends through the hole and wraps around the second section.

In some configurations, the second section may include a groove, wherein elastic cord wraps around the second section in the groove and the groove may prevent the elastic cord from moving vertically.

In some configurations, the fastener may include a hole in the overlapping part of the first section, and a fastener component that may extend from the overlapping part of the second section, wherein the fastener component may be inserted through the hole coupled the fastener component to the hole.

In some configurations, the fastener component may include a first portion that may extend from the overlapping part of the second section, and a second portion coupled to the first portion, wherein a width of the second portion is larger than a width of the first portion.

In some configurations, the fastener may include a bar coupled to the overlapping part of the first section and a hook coupled to the overlapping part of the second section.

In some configurations, the hook may be inserted into an opening of the bar to couple the hook to the bar.

In some configurations, the bar may be coupled to the overlapping part of the first section at an angle and the hook may be coupled to the overlapping part of the second section at the angle.

In some configurations, the sole portion may include at least one groove on an inner surface of the sole portion, wherein the at least one groove may be configured to guide water or debris out of the foot cover and/or allow airflow to dry off water in the foot cover.

### BRIEF DESCRIPTION OF THE DRAWINGS

These and other features, aspects, and advantages of the present disclosure are described with reference to the drawings of certain embodiments, which are intended to schematically illustrate certain embodiments and not to limit the disclosure.

FIG. 1A illustrates an example of a two-part foot cover in an open configuration.

FIG. 1B illustrates an example of a two-part foot cover in a closed configuration.

FIG. 1C illustrates a perspective view of an inner cavity of another example of a two-part foot cover.

FIG. 2A illustrates an example of an inner cavity of a first section of a two-part foot cover.

FIG. 2B illustrates an example of a cross-section of a two-part foot cover.

FIG. 3A illustrates an example of a two-part foot cover with a prosthetic foot halfway inserted into a foot cover.

FIG. 3B illustrates an example of a two-part foot cover with a prosthetic foot fully inserted into a foot cover.

FIG. 4A illustrates an example of a spot ring and an indentation of a two-part foot cover.

FIG. 4B illustrates an example of a two-part cover with holes.

FIG. 5 illustrates an example of an alternative two-part foot cover.

FIGS. 6A-6C illustrate examples of a bottom surface of a foot cover with grip feature(s).

FIG. 7 illustrates another example of a two-part foot cover with a seal.

FIG. 8A illustrates a partial perspective view of another example of a two-part foot cover with a seal.

FIG. 8B illustrates a partial top view of the example two-part foot over of FIG. 8B.

FIGS. 9A-9C illustrate example fasteners of a two-part foot cover of the present disclosure.

FIG. 10A illustrates an example of a two-part foot cover with a sockless prosthetic foot.

FIG. 10B illustrates an example of a two-part foot cover with another sockless prosthetic foot.

### DETAILED DESCRIPTION

These and other features, aspects, and advantages of the present disclosure are described with reference to the drawings of certain embodiments, which are intended to schematically illustrate certain embodiments and not to limit the disclosure.

### Example Features of Foot Cover for Prosthetic Foot

The present disclosure provides examples of a foot cover or cosmesis for a prosthetic foot allowing a user to more easily decouple the foot cover from the prosthetic foot. The foot cover may be more easily put on and removed from the prosthetic foot without tools. The foot cover examples disclosed herein can be waterproof. Additionally, the foot cover examples disclosed herein can have improved grip to reduce slipping, for example, in a wet environment.

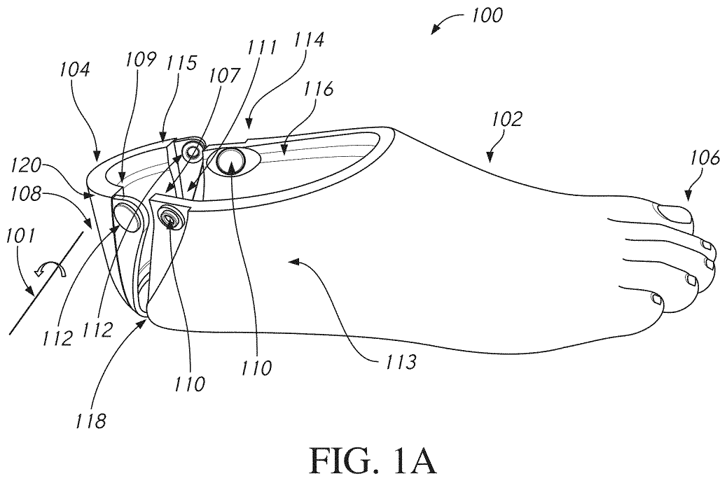

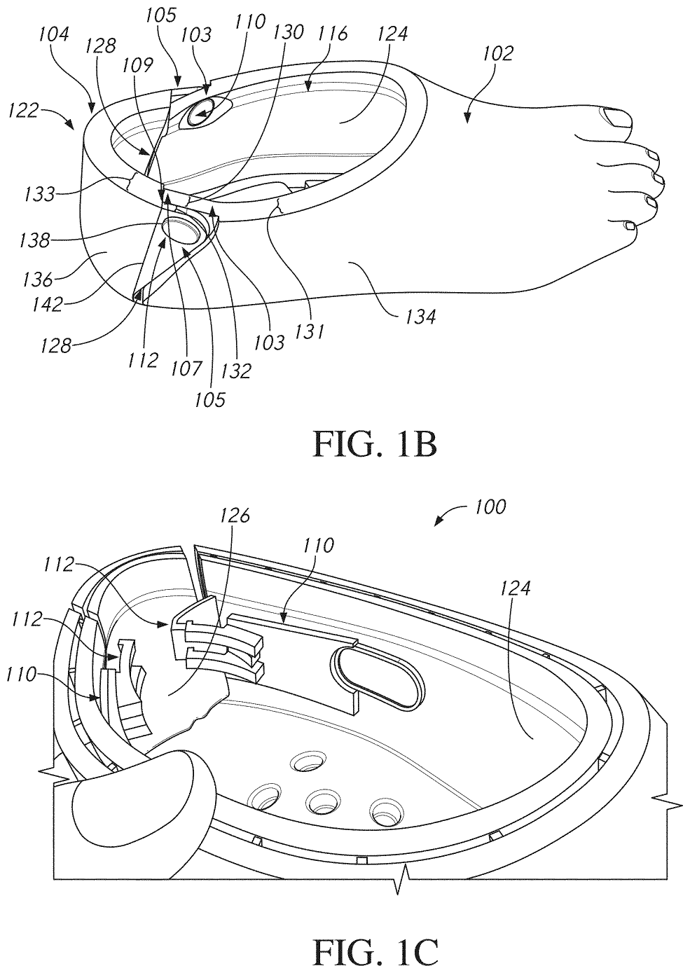

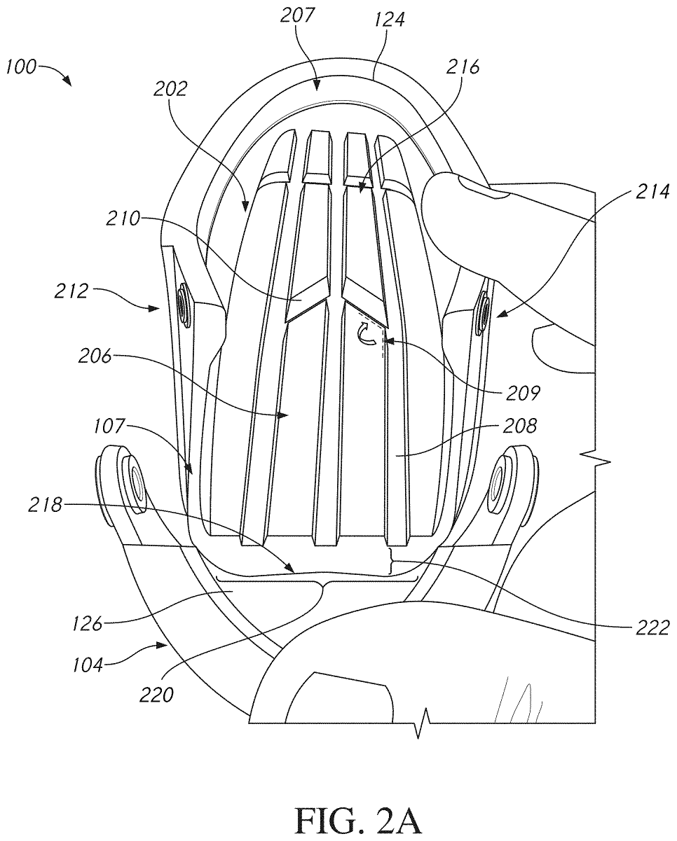

FIGS. 1A and 1B illustrate an example two-part foot cover 100. The foot cover 100 extends from a toe end 106 to a heel end 108. The foot cover 100 includes an opening 116 at or near an ankle location when a prosthetic foot is inserted into the foot cover 100. A prosthetic foot can be inserted into an inner cavity of the foot cover 100 via the opening 116. The foot cover 100 can be made of ethylene-vinyl acetate (EVA), polyurethane, thermoplastic polyurethane (TPU), injected rubber, compressed rubber, thermoplastic elastomer (TPE), or any other flexible and/or durable material used for foot covers.

The foot cover 100 can include a first section 102 and a second section 104. The first section 102 can include a portion of the foot cover 100 from the toe end 106 to a rear end 107 that is more rearward than an anterior end of the opening 116. In the illustrated example, the first section 102 can terminate at around a mid point of the overlapping portions 103 in the fore-aft direction. The second section 104 can include a remaining portion of the foot cover from an anterior end 109 that is more rearward than an anterior end of the overlapping portions 103 to the heel end 108 of the foot cover 100. In the illustrated example, the second section 104 can begin at around a mid point of the overlapping portions 103.

The second section 104 can be rotatably coupled to the first section 102 at the rear end 107 and the anterior end 109 with a hinge 118. The hinge 118 can be a live hinge or any other type of hinge. The hinge 118 can allow the second section 104 to rotate relative to the first section 102 around a transverse axis 101 (see FIG. 1A). The hinge 118 may be anywhere along the rear end 107 and the anterior end 109. In the illustrated example, the hinge 118 is at or near a heel end 108 of the foot cover 100. The hinge 118 can be located such that the second section 104 can flex easily backwards when in the open configuration, allowing the foot to be more easily removed, and more easily for a user to insert the foot into the foot cover 100 and move the second section 104 into the closed configuration. For example, the hinge 118 can be located such that the second section 104 can fit well in the hand of the user. The hinge 118 can be durable and does not easily tear due to repeated opening and closing the second section 104 relative to the first section 102.

The first section 102 and second section 104 can be releasably coupled, for example, by a fastener or any other suitable coupling mechanisms. The location of the fastener can be more anterior than the location of the hinge 118. The first section 102 can include one or more first fastener components 110 at or near the rear end 107. The one or more first fastener components 110 can be located at or near a top end 111 of the rear end 107. The one or more first fastener components 110 can be included on medial 114 and lateral 113 sides of the foot cover 100. The second section 104 can include one or more second fastener components 112 at the anterior end 109. The one or more second fastener components 112 can be complementary to the one or more first fastener components 110 to form a releasably connection. The one or more second fastener components 112 can be located at or near a top end 115 of the anterior end 109. The one or more second fastener components 112 can be included on medial 114 and lateral 113 side of the foot cover 100. The one or more first fastener components 110 and the one or more second fastener components 112 can include the male component or the corresponding female component of a fastener. The one or more first fastener components 110 and the one or more second fastener components 112 can be complementary components of the fastener. The fastener can include a clasp, snap, magnet, button, hook-and-loop fastener, an indentation and a raised surface, an elastic band and hook, or any other non-permanent mechanical fastener. In some embodiments, the fastening components can include a water repellent button that can be closed with a click.

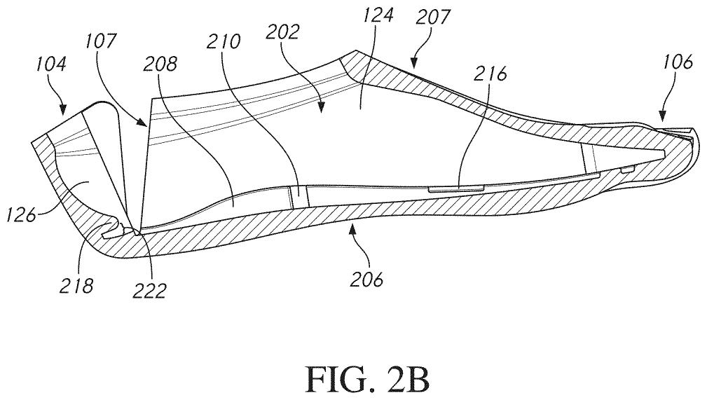

The second section 104 can be rotated between an open position or configuration 120, as shown in FIG. 1A, and a closed position or configuration 122, as shown in FIG. 1B. When the second section 104 is in a closed position 122, the fastener components 110, 112 can be coupled together to secure the second section 104 in a closed position 122 relative to the first section 102. The rear end 107 and the anterior end 109 can create a seal 128 (see FIG. 1B) when the second section 104 is in a closed position. In this illustrated example, the rear end 107 of the first section 102 and the anterior end 109 of the second section 104 can have complementary generally straight-lined edges on the medial and lateral sides of the foot cover 100 so that the seal 128 has a generally straight-lined configuration. The seal 128 can be a waterproof seal, such that water does not enter the foot cover from any position along the seal 128. In another embodiment, the seal 128 may include at least one hole on medial and lateral sides of the sole portion 206 (see FIG. 2A) due to the hinge placement. The at least one hole can be near the bottom of the foot cover 100. The at least one hole can be connected to a drainage groove (described in greater detail below) on the sole portion 206. In this embodiment, the at least one hole allows a user to drain water, dirt, or other debris from the foot cover when the second section 104 is in a closed position. The first section 102, the second section 104, and the hinge 118 can be made of the same material, or different materials.

The rear end 107 and the anterior end 109 can have complementary shapes. The overlapping portions 103, 105 can overlap when the second section 104 is in the closed position. The first section 102 and the second section 104 can include overlapping portions 103 and 105. The one or more first fastener components 110 and the one or more second fastener components 112 can be coupled at the overlapping portions 103, 105. The overlapping portions 103, 105 can each have a thickness smaller than a thickness of the wall of the foot cover 100 such that when the foot cover 100 is in the closed configuration, the overlapping sections can have a combined thickness substantially the same as the thickness of the wall of the foot cover 100. The overlapping portions 103, 105 can have a thickness 130, 132. As shown in FIG. 1B, the first section 102 can have a thickness 131, and the second section can have a thickness 133. Thickness 131 and 133 can be the same. Thickness 130 and 132 can be smaller than thickness 131 and 133 such that thickness 130 and 132 added together (that is, in the closed configuration) are generally the same as thickness 130 and 132. In this way, when the second section 104 is in a closed position 122, an inner surface 124 of first section 102 and an inner surface 126 of the second section 104 are generally flush with each other. In the same way, when the second section 104 is in a closed position 122, an outer surface 134 of first section 102 and an outer surface 136 of the second section 104 are flush with each other.

The overlapping portion 105 can flex or bend along line 142 to allow a user to rotate the second section 104 from an open position 120 to a closed position 122 and vice versa without the overlapping portion 105 hitting the overlapping portion 103. Having the overlapping portion flex or bend along line 142 also allows a user to couple or decouple the components of the fastener 110, 112.

FIG. 1C illustrates an example hinged foot cover 100 with an alternative fastener placement. The foot cover 100 in FIG. 1C can have any of the features of the foot 100 in FIGS. 1A and 1B with the difference described with reference to FIG. 1C. In FIG. 1C, the fastener components 110, 112 can be located on the inner surface 124, 126 of the first section 102 and the second section 104 respectively. The alternative fastener placement hides the fastener components 110, 112 inside the foot cover so the components of the fastener 110, 112 cannot be seen, improving the overall aesthetic of the foot cover 100. The components of the fastener 110, 112 may include grip hooks. The first and second sections in FIG. 1C are divided at a location more rearward than as shown in FIGS. 1A and 1B. The relative lengths of the first and second sections can be varied.

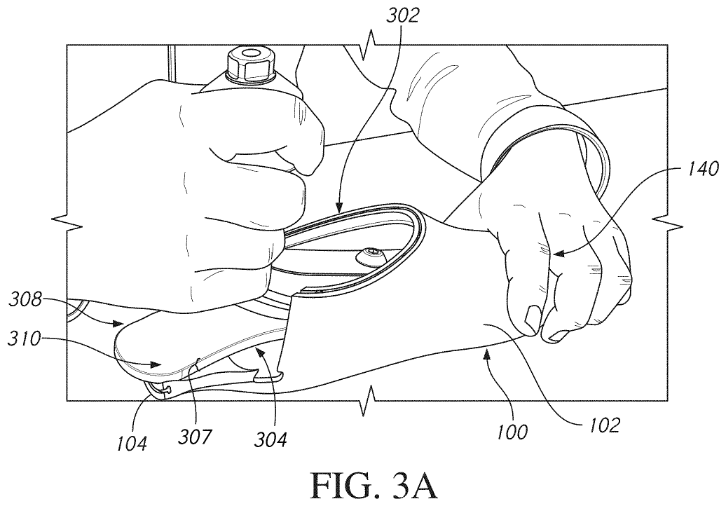

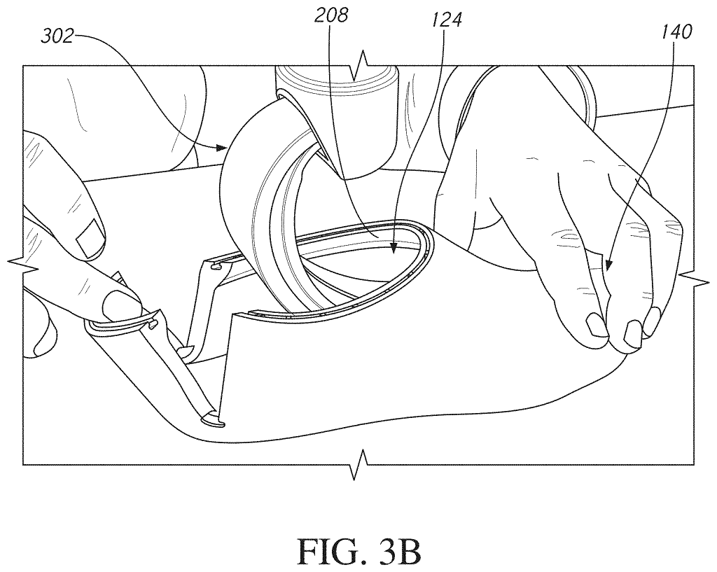

FIG. 2A illustrates the inner cavity 202 of the foot cover 100. The inner cavity 202 of the foot cover 100 can have generally the same shape as a prosthetic foot that can be received by the inner cavity 202 of the foot cover 100, such as the prosthetic foot 302 shown in FIG. 3A. The inner cavity 202 can be defined by the inner surface 124 and the inner surface 126. The inner surface 124 can include a sole portion 206, and a top portion 207. The sole portion 206 can have generally the same shape as a sole surface of a prosthetic foot that can be received in the foot cover 100, for example, the sole surface 304 of the prosthetic foot 302 shown in FIG. 3A. The top portion 207 can be coupled to the sole portion 206 along an edge or periphery of the sole portion 206 and can form the top of the foot cover 100. An upper edge of the top portion 207 can form the opening 116 leading to the inner cavity 202. The sole portion 206 can include one or more grooves 208. The grooves 208 can run from the rear end 107 to the toe end 106 of the first section. The grooves 208 can run generally parallel to a length of the foot cover 100. The grooves 208 can be generally symmetric about a central axis along the length of the foot cover 100. For example, as shown in FIG. 2A, the sole portion 206 can include three grooves 208. The groove 208 in the middle can be generally aligned with the central axis and the other two grooves 208 can be symmetric about the central axis. The grooves 208 can have varying depths along the length of the foot cover 100. The grooves 208 may be deeper at thicker regions of the sole portion 206.

The sole portion 206 can include one or more cross-grooves 210. The cross-grooves 210 can run from a lateral side 212 of the sole portion 206 to the medial side 214 of the sole portion 206. The cross grooves 210 can intersect the grooves at an angle 209. The angle 209 can be any angle between 0 degrees and 180 degrees. The cross-grooves 210 can have any of the features of the grooves 208 except in the directions of the cross-grooves 210. The grooves 208 and the cross-grooves 210 can guide water, dirt, or other debris out of the foot cover 100 and/or allow airflow to dry off water in the foot cover 100. The grooves 208 and cross-grooves 210 can also facilitate more even shrinkage of the foot cover 100, which may have uneven thickness in the sole portion 206, during a manufacturing process.

Optionally, the sole portion 206 can include at least one recess 216. The at least one recess 216 can be of generally the same shape and location as at least one feature of the prosthetic foot that can be received by the foot cover 100. For example, the prosthetic foot 302 includes at least one bolt for coupling the foot members. The shape and location of the at least one recess 216 can correspond to the at least one bolt of the prosthetic foot 302. The recess 216 can be generally circular or oval with a dimension that can accommodate the at least one bolt. The at least one recess 216 can be located in a metatarsal region of the sole portion 206. The at least one bolt of the prosthetic foot 302 can be received by the at least one recess 216 when the prosthetic foot 302 is inserted into the foot cover 100. The recess can allow for substantially the entire bottom surface 304 of the prosthetic foot 302 to contact the sole portion 206. The sole portion 206 can optionally include a recess at a different location and/or with a different shape, or additional recess(es) of various shapes and locations depending on any downwardly protruding features of the prosthetic foot received by the foot cover 100 such that the bottom surface of the prosthetic foot can form a better contact with the sole portion 206.

As shown in FIG. 2B, the inner surface 126 of the second section 104 can include a securement feature 218. The securement feature 218 can be a raised portion of the inner surface 126. For example, the securement feature 218 can be in the form of a lip. The securement feature 218 can be generally the width 220 of the inner surface and can be a height 222 from the bottom of the inner surface. The height 222 can be substantially the same as the thickness of a heel portion of a bottom foot member of the prosthetic foot that can be received by the foot cover 100, for example, the thickness 307 of the bottom member 308 of the prosthetic foot 302 as shown in FIG. 3A. When the second section 104 is in a closed position 122, the securement feature 218 can extend over the heel portion of the bottom member 308, locking the bottom member 308 into the foot cover 100. The securement feature 218 can provide a downward force on the top surface 310 of the bottom member 308 securing the prosthetic foot 302 in the foot cover 100. When the second section 104 is in the open configuration, the securement feature 218 can ensure that the foot is in place before pushing the hinge 118 up and engage the two fastener components. The hinge 118 and the securement feature 218 can make it easier for the foot to fit into the inner cavity 202 without a user having to force the second section 104 into the closed position or configuration.

FIGS. 3A and 3B illustrate an example of a prosthetic foot 302 being inserted into the hinged foot cover 100. A user can insert the prosthetic foot 302 into the foot cover 100 with the second section 104 in an open position 120. A toe end (not shown) of the prosthetic foot 302 can be inserted into the first section 102 of the foot cover 100. The toe end of the prosthetic foot 302 can rest generally against the toe end 106 of the foot cover 100. The top portion 207 of the inner surface 124, including the securement feature 218, can secure the prosthetic foot against the sole portion 206 of the inner surface 124. When the prosthetic foot 302 is inserted completely into the inner cavity 202, the second section 104 may be rotated by a user from the open position to the closed position or configuration.

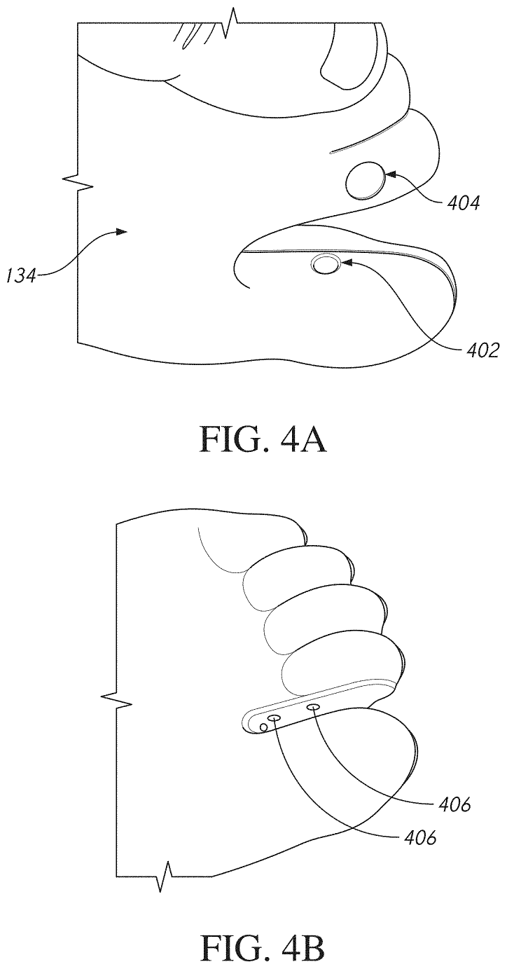

FIG. 4A illustrates a spot 402 and a ring 404. The foot cover 100 can include at least one spot 402 and/or at least one ring 404. The spot 402 and/or ring 404 can be located anywhere on the outer surface 134 of the foot cover 100. In some configurations, the spot 402 and/or ring 404 may be more concentrated or solely in the first section 102. The spot 402 can be a generally circular shaped indentation on the outer surface 134. The ring 404 can be a generally circular ring-shaped indented portion of the outer surface 134. Although the spot 402 is described as generally circular, the spot 402 can be any shape. Furthermore, the ring 404 can be any ring shape. The spot 402 and/or ring 404 can be a guide for where a user can drill a hole in the foot cover 100. Examples of the holes 406 are shown in FIG. 4B. The hole can allow for water, dirt, sand or other debris to be drained from the foot cover 100. The hole can also allow a user to flush water through the foot cover 100 to clean the inner cavity 202 without having to remove the prosthetic foot 302 from the foot cover 100.

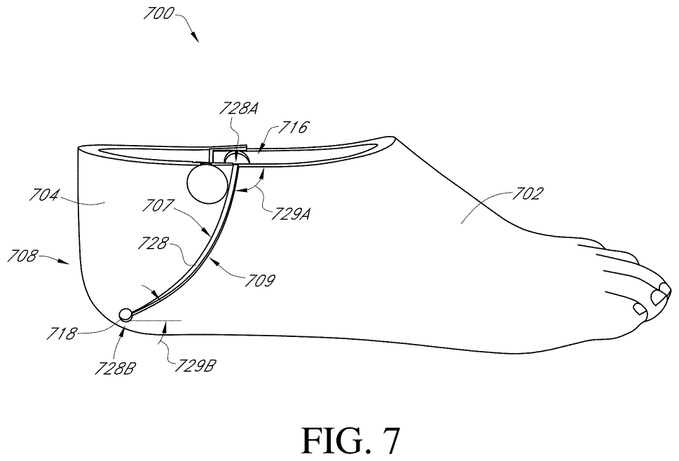

FIG. 7 illustrates an example hinged foot cover 700 with a seal 728. The foot cover 100 in FIGS. 1A-4B can have any of the features of the foot cover 700 in FIG. 7, and the foot cover 700 in FIG. 7 can have any of the features of the foot cover 100 in FIGS. 1A-4B except the differences described with reference to FIG. 7. As described above, in the foot cover 100, the seal 128 can have a generally straight configuration. In FIG. 7, the seal 728 can include a curved configuration. In this illustrated example, the rear end 707 of the first section 702 and the anterior end 709 of the second section 704 can have complementary generally curved edges on the medial and lateral sides of the foot cover 700. The seal 728 can include a top portion 728A and a bottom portion 728B. The top portion 728A of the seal 728 can be a location where the seal 728 intersects an opening 716 of the foot cover 700. The bottom portion 728B of the seal 728 can be a location where the seal 728 intersects the hinge 718 near the bottom of the foot cover 700.

The seal 728 can curve along a length of the seal 728 from the bottom portion 728B of the seal 728 to the top portion 728A of the seal 728. The seal 728 can curve rearwardly (that is, towards the heel end 708 of the foot cover 700) from the bottom portion 728B of the seal 728 to the top portion 728A of the seal 728. Alternatively, the seal 728 can curve rearwardly (that is, towards the heel end 708 of the foot cover 700) from the top portion 728A of the seal 728 to the bottom portion 728B of the seal 728.

The top portion 728A of the seal 728 can form a first angle 729A with a transverse plane (i.e., a horizontal plane generally parallel to the bottom of the foot cover 700) and the bottom portion 728B of the seal 728 can form a second angle 729B with the transverse plane. The first angle 729A can be larger than the second angle 729B such that the seal 728 is curved at a less steep slope near the bottom portion 728B than the top portion 728A. Alternatively, the second angle 729B can be smaller than the first angle 729A such that the seal 728 is more vertical (i.e., more perpendicular to the transverse plane) near the top portion 728A than the bottom portion 728B.

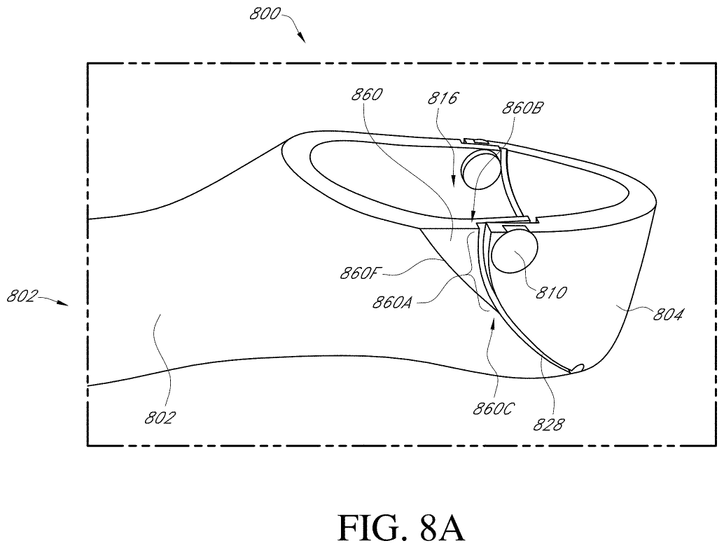

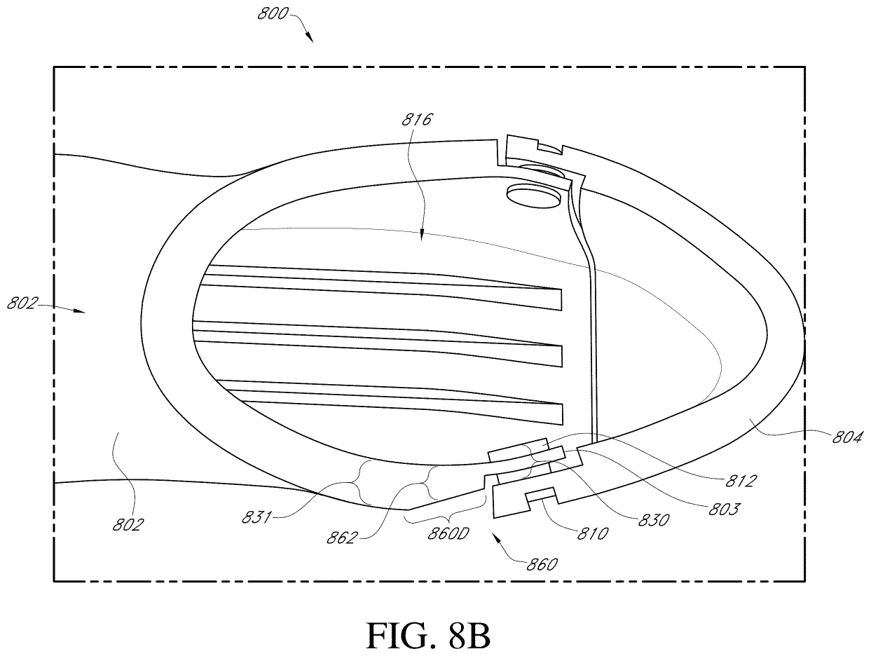

FIGS. 8A-8B illustrate an example hinged foot cover 800. The foot cover 100, 700 in FIGS. 1A-4B, 6A-6C and 7 can have any of the features of the foot cover 800 in FIGS. 8A-8B and the foot cover 800 in FIGS. 8A-8B can have any of the features of the foot covers 100, 700 in FIGS. 1A-4B, 6A-6C and 7 except the differences described with reference to FIGS. 8A-8B. In FIG. 8A, the foot cover 800 can include a first section 802 and a second section 804. The first section 802 can include a tapered portion 860. The tapered portion 860 can be located at a portion of the first section 802 near the seal 828. The tapered portion 860 can have a thickness 862. The thickness 862 can increase (for example, gradually increase) from a thickness 830 of the overlapping portion 803 to a thickness 831 of the first section 802.

The tapered portion 860 can include a height 860A from a top end 860B of the tapered portion 860 and a bottom end of the tapered portion 860. The top end 860B can be located at an opening 816 at or near an ankle location so that a prosthetic foot can be inserted into the foot cover 800. The bottom end 860C of the tapered portion 860 can be located closer to the bottom surface of the foot over 800, for example, between the opening 816 and the bottom surface of the foot cover 800. Optionally, the tapered portion 860 can extend along substantially an entire height of the foot cover 800.

The tapered portion 860 can include a width 860D extending generally along a length of the foot cover 800. The width 860D can include a distance from the seal 828 to a front edge 860F of the tapered portion 860. The front edge 860F of the tapered portion 860 can be curved or alternatively generally straight such that the width 860D of the tapered portion 806 can decrease (for example, gradually decrease) from the top end 860B toward the bottom end 806C. Optionally, the tapered portion 860 can have a generally triangular shape.

The tapered portion 860 can allow a user to more easily grip onto fastener components 810, 812. In this way, the user can more easily couple or decouple the fastener components 810, 812 for faster donning and removal of the foot cover 800.

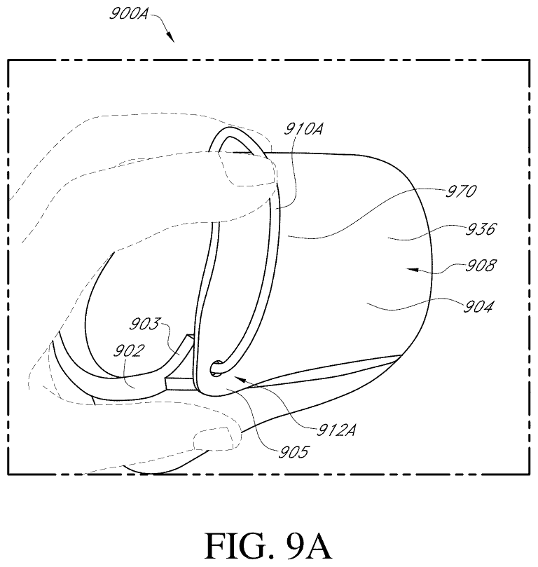

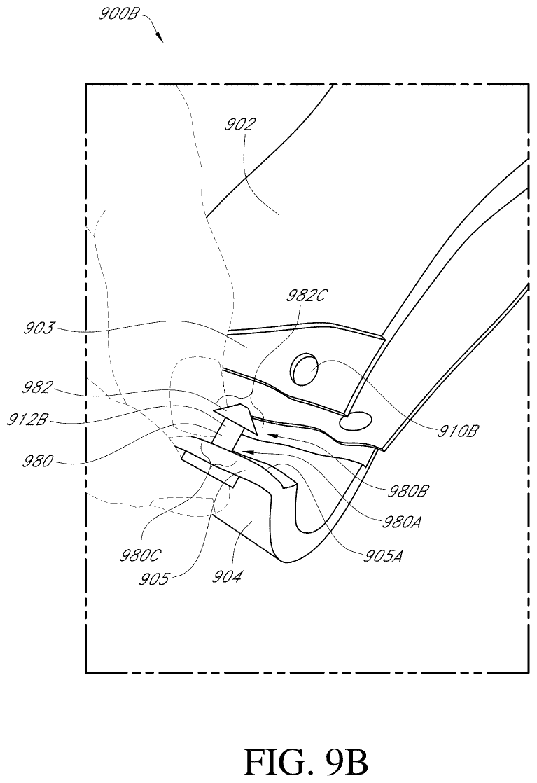

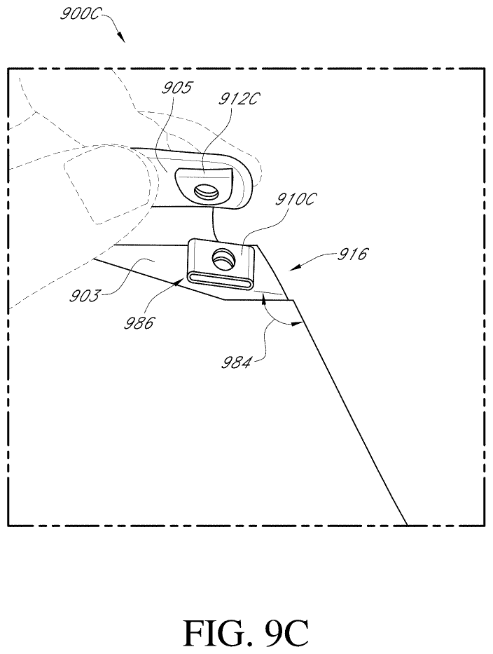

In addition to the fasteners disclosed with reference to the foot 100, 700, 800, FIG. 9A-9C illustrate a hinged foot covers 900A, 900B, 900C with different types fasteners. The foot covers 100, 700, 800 can have any of the features of the foot covers 900A, 900B, 900C; and foot covers 900A, 900B, 900C in FIGS. 9A-9C can have any of the features of the foot covers 100, 700, 800 in FIGS. 1A-4B, 6A-6C, 7 and 8A-8B except the differences described with references to FIG. 9A-9C. In FIG. 9A, the fastener can include fastener components 910A, 912A. The fastener component 912A can include an opening in an overlapping portion 905 of a second section 904 of the prosthetic foot 900A. The fastener component 910A can include a rubber band or an elastic band coupled to an overlapping portion 903 of a first section 902. The fastener component 910A can extend through the fastener component 912A and wrap around the second section 904 (for example, the heel end 908) of the foot cover 900A. Optionally, the fastener component 910A can wrap around the second section 904 of the foot cover 900A in a groove 970 in an outer surface 936 of the second section 904. The groove 970 can prevent the fastener component 910A from sliding or moving vertically along the outer surface 936 of the second section 904. Tension in the fastener component 910A can inhibit rotation of the second section 904 from a closed position to an open position.

In FIG. 9B, the fastener can include fastener components 910B, 912B. The fastener component 910B can include an opening in the overlapping portion 903 of the first section 902. The fastener component 912B can include a first portion 980 and a second portion 982. The first portion 980 can extend from an inner surface 905A of the overlapping portion 905. The first portion 980 can include a first end 980A and a second end 980B opposite the first end 980A. The first end 980A can be coupled to the inner surface 905A of the overlapping portion 905. The second portion 982 can be coupled or attached to the second end 980B of the first portion 980.

The first portion 980 can include a cylindrical shape, a rectangular prism shape, a triangular prism shape, and/or any other shape. The second portion 982 can include a profile shape when viewed from the side. The profile shape can include a triangle, a trapezoid, semicircle, and/or any other shape. In the illustrated example, the second portion 982 can have a dome shaped.

The first portion 980 and the second portion 982 each can include a width 980C, 982C. The width 982C of the second portion 982 can be larger than the width 980C of the first portion 980. The width 982C of the second portion 982 can be larger at an end of the second portion 982 coupled to the first portion 980 than the width 982C of the second portion 982 at an end opposite the end coupled to the first portion 980. The profile shape of the second portion 982 can allow ease of insertion of the second portion 982 through the fastener component 910B and prevent the second portion 982 from being removed from the fastener component 910B. In this way, the fastener component 912B can be inserted into the fastener component 910B so the first portion 980 can extend through the fastener component 910A and the second portion 982 can contact an inner surface of the overlapping portion 903 to couple the fastener portion 912B to the fastener component 910B.

In FIG. 9C, the fastener can include fastener component 910C, 912C, which can include a hook and bar fastener. The fastener component 910C can include a bar coupled to the overlapping portion 903. The fastener component 912C can include a hook coupled to the overlapping portion 905. The fastener component 912C can be inserted into the fastener component 910C to couple the fastener component 912C to the fastener component 910C. The fastener component 910C can be coupled to the overlapping portion 903 at an angle 984 and/or the fastener component 912C can be coupled to the overlapping portion 905 at the angle 984. The angle 984 can include an angle between an opening 986 of the bar and a top surface of the foot cover 900C at an opening 916 of the foot cover 900C. The angle 984 can allow a user to more easily insert the fastener component 912C into the fastener component 910C. The angle 984 can prevent the fastener component 912C from sliding out of the fastener component 910C.

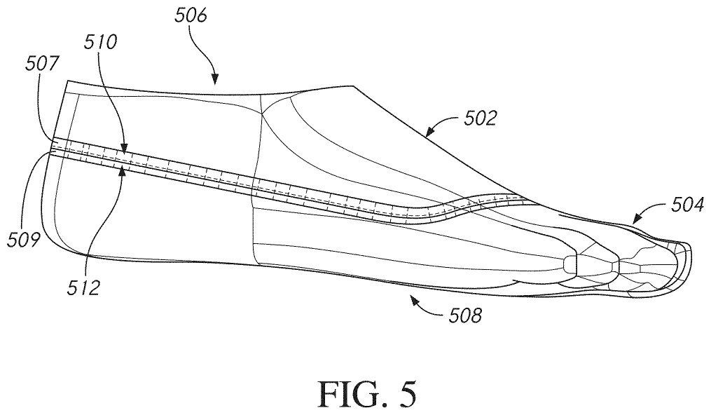

FIG. 5 illustrates an alternative example two-part foot cover 500. The foot cover 500 can include any of the features of the foot cover 100 disclosed herein except the differences described with reference to FIG. 5. The foot cover 500 can include a first section 502 and a second section 504. The first section 502 can include a portion of the foot cover from an ankle end 506 to a generally mid-foot border 507. The second section 504 can include a remaining portion of the foot cover from a generally mid-foot border 509 to a heel end 508 of the foot cover 500. The mid-foot border 507 and the mid-foot border 509 can have complementary shapes. The first section 502 can include one or more first magnets 510 around the mid-foot border 507. The second section 504 can include one or more second magnets 512 around the mid-foot border 509. The magnet(s) 510, 512 can be a single magnet or a plurality of magnets arranged in a ring around the mid-foot border 507, 509. Alternatively, the one or more magnets 510, 512 can extend at least partially around the mid-foot border 507, 509 respectively. In one embodiment, the magnets partially along the mid-foot border 507 and the magnets partially along the mid-foot border 509 can be arranged in alternating positions so that when the first and second sections 502, 504 are coupled along the mid-foot borders 507, 509, the magnets along on the mid-foot border 507 and the mid-foot border 509 form a complete ring. The first and second rings of magnets 510, 512 can have opposite polarities so that the first and second sections 502, 504 can be connected at the mid-foot portions 507, 509 to form the foot cover 500. In some embodiments, the connection between the first and second rings of magnets 510, 512 is waterproof. In some embodiments, the mid-foot borders 507, 509 can be located at different parts of the foot cover 500 than as illustrated in the non-limiting example of FIG. 5. Although the generally mid-foot border 507 is illustrated to be near a mid point along a height of the foot cover 500, the generally mid-foot border 507 can be located anywhere along the height of the foot cover 500.

Alternative or in addition to using the rings of magnets 510, 512, the first and second sections 502, 504 of the foot cover 500 can be mechanically connected by other suitable quick release mechanisms, such as any mechanism in which two components connect with a click.

The foot cover examples disclosed herein, for example, the foot cover 100, 500, 700, can include grip features to reduce slipping. Prosthetic users are prone to slipping on wet surfaces, for example, in a bathroom, shower, a swimming pool area, floor with spilled liquid, the roads during rain or snow, and the like. Existing foot covers can be slippery in a wet environment. The tendency to slip can be due to a variety of reasons, for example, inadequate contact between the bottom of the foot covers and the ground (or any other contact surface) to provide sufficient friction against slipping, especially in wet conditions, and/or the material of the foot covers (for example, EVA, polyurethane, TPU, injected rubber, compressed rubber, TPE, or any other flexible or elastic material) being hard, therefore less capable of conforming to the contact surface compared to a more compliant material. The grip features disclosed herein can improve grip of the foot covers.

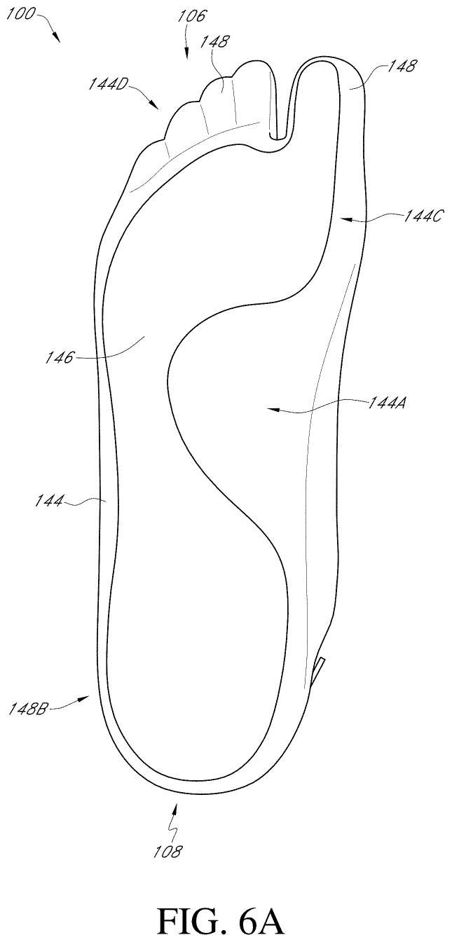

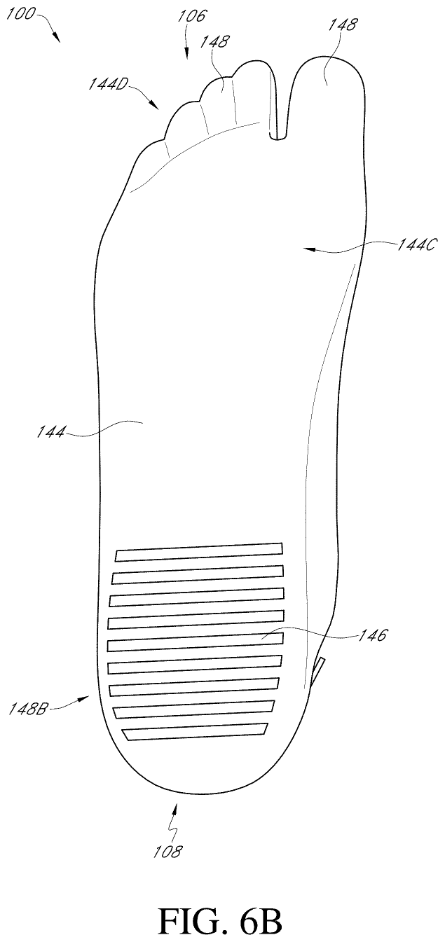

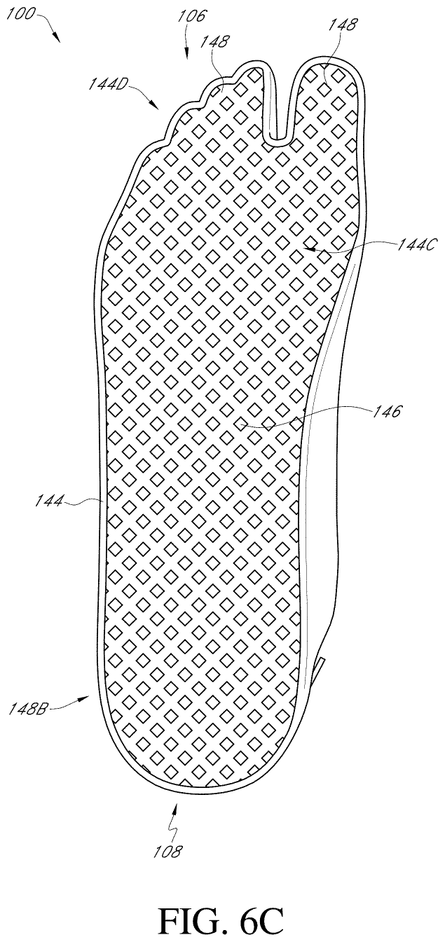

Such grip features are illustrated in FIGS. 6A-6C using the foot cover 100 as an example. However, the grip features shown in FIGS. 6A-6C can be incorporated in any other foot cover examples disclosed herein. FIGS. 6A-6C illustrate a bottom surface 144 of the foot cover 100. The bottom surface 144 can include grip features 146. The grip features 146 can be located on at least a portion of or throughout substantially the entire bottom surface 144 to provide increased friction between the foot cover 100 and a contact surface.

As best in FIG. 6A, the grip features 146 can be formed by sandblasting at least a portion of the bottom surface 144 or coupling a sandblasted piece of material removably or permanently to the bottom surface 144. The sandblasted piece of material can be the same material as a material the bottom surface 144, or the sandblasted piece of material can be a different material from the material of the bottom surface 144.

As shown in FIG. 6B, the grip features 146 can include a plurality of cuts, micro cuts and/or indentations 146A (for example, directly) on the bottom surface 144. The plurality of cuts, micro cuts and/or indentations 146A may also be incorporated into a piece of material (such as shown in FIG. 6A) coupled to the bottom surface 144. The plurality of cuts, micro cuts, and/or indentations 146A can push water away from the bottom surface 144. The plurality of cuts, micro cuts, or indentations can increase contact between the bottom surface 144 and a contact surface.

Optionally, each of the plurality of cuts, micros cuts and/or indentations 146A can be parallel or substantially parallel to the rest of the plurality of cuts, micro cuts or indentations 146A. Alternatively, at least some of the cuts, micro cuts, and/or indentations 146A may be at a nonparallel angle with one another. the plurality of cuts, micro cuts and/or indentations 146A can resemble wrinkled skin. The plurality of cuts, micro cuts and/or indentations 146A can also form a pattern. For example, the pattern of cuts, micro cuts or indentations 146A can be a knurling pattern, a checker pattern, a plurality of shapes, and/or any other pattern. The plurality of shapes can include squares, circles, triangles, diamonds, ellipses, rectangles, and/or any other shape.

The plurality of cuts, micro cuts and/or indentations 146A can from an angle 150 with a longitudinal axis 151 that extends from the toe end 106 of the foot cover 100 to the heel end 108 of the foot cover 100. The angle 150 can be between 0 degrees and 180 degrees, or between 0 degrees and 90 degrees. For example, the angle 150 can be 0 degrees, 5 degrees, 10 degrees, 15 degrees, 20 degrees, 25 degrees, 30 degrees, 35 degrees, 40 degrees, 45 degrees, 50 degrees, 55 degrees, 60 degrees, 65 degrees, 70 degrees, 75 degrees, 80 degrees, 85 degrees, 90 degrees, and/or any value between the aforementioned values.

As shown in FIG. 6C, the grip feature 146 can include a tread pattern 146B. The tread pattern 146B can be part of the bottom surface 144 (i.e., molded on or into the bottom surface 144), or the tread pattern 146B can be molded on or into a piece of material coupled to the bottom surface 144. The tread pattern 146B can be grid-shaped or cross-hatched. The tread pattern 146B can also include a knurling pattern, a checker pattern, a plurality of shapes, and/or any other pattern. The plurality of shapes can include squares, circles, triangles, diamonds, ellipses, rectangles, and/or any other shape. The tread pattern 146B can also include one or more studs, bars, lugs, and/or any other extrusion.

The grip features 146 can be on a portion of the bottom surface 144 or cover a portion of the bottom surface 144. The grip features 146 can extend along a portion of the length of the bottom surface 144 and/or a portion of the width of the bottom surface 144. The grip features 146 can be on or cover a portion of the bottom surface 144 that contacts a walking surface. The grip features 146 can cover, be on, or extend along substantially the entire bottom surface 144. The grip features 146 can extend along substantially an entire length of the bottom surface 144 and/or an entire width of the bottom surface 144.

As shown in FIG. 6A, a portion of the bottom surface 144 can be without the grip features 146, for example, the arch region 144A of the bottom surface 144. The arch region 144A without the grip features 146 can be a portion of the bottom surface 144 that does not typically contact the walking surface. The grip features 146 can be on or cover the entire bottom surface 144 except the arch portion 144A. For example, the sandblasted piece of material can extend to the bottom surface at least under the big toe, the forefoot region, a lateral portion of the arch region, and the heel region.

As shown in FIG. 6B, the grip features 146 can be on or cover a heel region 144B of the bottom surface 144. The grip features 146 can be on or cover a forefoot region 144C of the bottom surface. The grip features 146 can be on or cover the heel region 144B of the bottom surface 144 and the forefoot region 144C of the bottom surface 144.

As shown in FIGS. 6A-6C, the toe region 106 of the foot cover 100 can include toes 148. The toes 148 can be a portion of the foot cover 100 shaped and/or sized to resemble toes of a foot. The grip features 146 can be on or cover a toe region 144D of the bottom surface 144. Alternatively, the grip features 146 can be on or cover some toes of the toe region 144D of the bottom surface 144.

As shown in FIG. 6A, the grip features 146 can be on or cover at least the big toe 148. As shown in FIG. 6B, the grip features 146 ay not cover the toe region 144D of the bottom surface 144. As shown in FIG. 6C, the grip features 146 can cover all the toes of the toe region 144D. Alternatively, the grip features 146 may cover any of the toes or combination of the toes of the toe region 144D.

The grip features 146 can include or be made of ethylene-vinyl acetate (EVA), polyurethane, thermoplastic polyurethane (TPU), thermos plastic rubber injected rubber, compressed rubber, leather, Vibram®, cork, nitrile, latex, and/or any other material.

### Example Sockless Prosthetic Foot

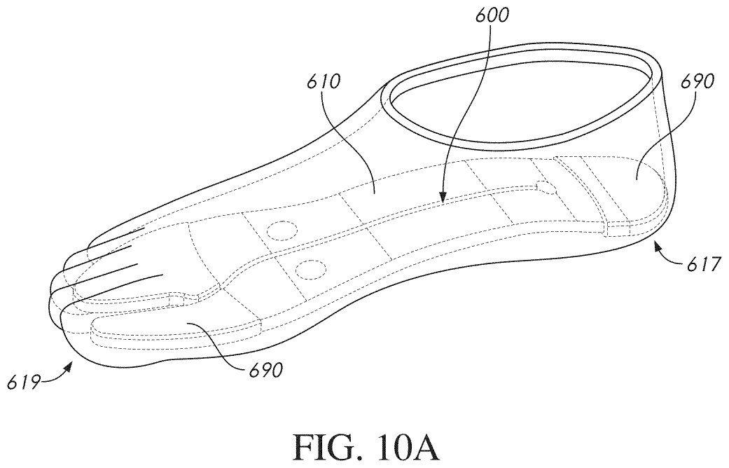

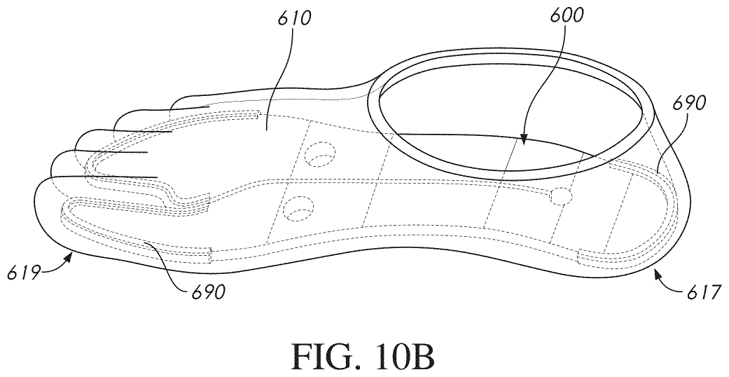

Typically, a sock is worn over the prosthetic foot before the foot is placed inside a foot cover to improve fit between the prosthetic foot and an inner cavity of the foot cover, reduce noise, and/or to reduce wear and tear of the foot cover by movements of the foot during which the carbon fiber foot member can cut the foot cover. In addition, the carbon fiber foot member can be slippery over the foot cover and cause the amputee to fall easily when used without the sock. In some embodiments, a prosthetic foot can be placed in a foot cover without a sock lining between the prosthetic foot and the foot cover. FIGS. 10A and 10B illustrate an example prosthetic foot 600 that can be inserted into the foot cover without a sock. One or more grip pads 690 can be added to a bottom or lower surface and/or a top surface of a foot member 610 to improve coupling between the prosthetic foot 600 and the foot cover 100 or the foot cover 500. The grip pads can be added under and/or above a toe region, a metatarsal region, and/or a heel region of the foot member 610, or anywhere experiencing high strain during heel strike and/or toe off. As shown in FIG. 10A, one or more grip pads 690 can be added to a top surface of the foot member 610. The grip pad(s) 690 on the top surface can be located at or near a toe region 619 of the foot member 610. As shown in FIG. 10B one or more grip pads 690 can be added to a bottom (or sole) surface of the foot member 610. The grip pad(s) 690 on the bottom surface can be located at or near a toe region 619 and/or a heel region 617 of the foot member 610. The grip pads 690 can extend around the edges in the toe region 619 and/or the heel region 617. As shown in FIG. 10A, the grip pads 690 can be added to the top and bottom surfaces and around the edges near the toe region 619 and the heel region 617. As shown in FIG. 6B, the grip pads 690 can be added to the bottom surface and around the edges near the toe region 619 and the heel region 617. In some embodiments, the grip pad 690 can include a diamond-structured pattern. The grip pad 690 can improve precompression by the prosthetic foot 600 to the foot cover 100. The grip pad 690 can improve gripping into the foot cover material, (for example, Ethylene-vinyl acetate, also known as EVA). The grip pad 690 can improve locking of the prosthetic foot inside foot cover 100 or foot cover 500, which can prevent noise during movement of the prosthetic foot. Alternatively or additionally, one or more grip pads can be added to the inner sole surface of foot cover 100 or foot cover 500.

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this invention may comprise, additional to its essential features described herein, one or more features as described herein from each other embodiment of the invention disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a sub combination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise. Additionally, as used herein, “gradually” has its ordinary meaning (e.g., differs from a non-continuous, such as a step-like, change).

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A foot cover for a prosthetic foot, the foot cover comprising:
a first section; and
a second section configured to be removably coupled to the first section to form the foot cover that includes a sole portion and a top portion connected to the sole portion along a periphery of the sole portion to define an inner cavity configured to receive the prosthetic foot, an upper edge of the top portion forming an opening leading to the inner cavity,
wherein the first and second sections form a releasable connection at a location near the upper edge of the top portion.

2. The foot cover of claim 1, wherein the foot cover is made of a waterproof material.

3. The foot cover of claim 2, wherein the waterproof material comprises EVA, polyurethane, TPU, injected rubber, TPE, or compressed rubber.

4. (canceled)

5. (canceled)

6. The foot cover of claim 1, further comprising at least one fastener configured to releasably couple the second section to the first section.

7. The foot cover of claim 5, wherein the at least one fastener is on an outer surface of the foot cover.

8. The foot cover of claim 5, wherein the at least one fastener is on an inner surface of the foot cover.

9. The foot cover of claim 5, wherein the at least one fastener comprises a clasp, snap, magnet, button, hook-and-loop fastener, an indention and raised surface, or an elastic band and hook.

10. A system comprising the foot cover of claim 1 and the prosthetic foot, wherein the prosthetic foot comprises a grip pad at one or both of a heel portion or a toe portion of the foot prothesis.

11. A foot cover for a prosthetic foot, the foot cover comprising:
a first section;
a second section rotatably coupled to the first section at a hinge to form the foot cover that includes a sole portion and a top portion connected to the sole portion along a periphery of the sole portion to define an inner cavity configured to receive the prosthetic foot, an upper edge of the top portion forming an opening leading to the inner cavity; and
at least one fastener configured to couple the first section to the second section at a second location away from the hinge,
wherein the first section comprises a front section and the second section comprises a heel section.

12. The foot cover of claim 10, wherein the hinge comprises a live hinge.

13. The foot cover of claim 10, wherein the sole portion comprises at least one groove on an inner surface of the sole portion, wherein the at least one groove is configured to guide water or debris out of the foot cover and/or allow airflow to dry off water in the cosmesis.

14. The foot cover of claim 10, wherein the at least one fastener comprises a clasp, snap, magnet, button, hook-and-loop fastener, an indention and raised surface, or an elastic band and hook.

15. The foot cover of claim 10, wherein the second section comprises a raised portion on an inner surface of the second portion, the raised portion configured to secure a heel portion of the prosthetic foot when the prosthetic foot is inserted into the first section.

16. (canceled)

17. (canceled)

18. (canceled)

19. The foot cover of claim 10, wherein the second location is more anterior than a location of the hinge.

20. The foot cover of claim 10, wherein the first and second sections comprise overlapping parts when the second section is fastened to the first sections.

21. A foot cover for a prosthetic foot, the foot cover comprising:
a bottom section configured to receive the prosthetic foot, the bottom section comprising a top opening; and
a top section configured to cover a top of the prosthetic foot, and secure the prosthetic foot in the bottom section, the top section comprising a bottom opening,
wherein the top opening is removably coupled to the bottom opening.

22. The foot cover of claim 21, wherein the bottom section comprises an inner surface, and wherein the inner surface comprises at least one groove configured to guide water or debris out of the cosmesis and/or allow airflow to dry off water in the cosmesis.

23. The foot cover of claim 21, wherein the top opening and the bottom opening each comprise a perimeter, each perimeter comprising one component of a fastener.

24. The foot cover of claim 23, wherein the fastener comprises a quick release fastener.

25. The foot cover of claim 23, wherein the perimeter of the top opening comprises a first one or more magnets, and the perimeter of the bottom opening comprises a second one or more magnets, the first and second one or more magnets having opposite polarities.
